(19) United States
(12) Patent Application Publication
(10) Pub. No.: US 2024/0075032 A1
(43) Pub. Date: Mar. 7, 2024

(54) A Pharmaceutical Composition for Treating Cancer

(71) Applicants: XUANZHU BIOPHARMACEUTICAL CO., LTD., Shijiazhuang, Hebei (CN); SHANDONG XUANZHU PHARMA CO., LTD., Jinan, Shandong (CN)

(72) Inventors: Yanjun XU, Shijiazhuang, Hebei (CN); Jiakui LI, Jinan, Shandong (CN)

(21) Appl. No.: 18/270,495

(22) PCT Filed: Dec. 31, 2021
(86) PCT No.: PCT/CN2021/143483

§ 371 Date: Jun. 30, 2023

(30) Foreign Application Priority Data
Dec. 31, 2020 (CN) 202011632660.3

(51) Int. Cl. A61K 31/506; A61K 31/565; A61P 35/00

(52) U.S. Cl. CPC A61K 31/506; A61K 31/565; A61P 35/00

(57) ABSTRACT

The present application belongs to the technical field of medicines, and specifically relates to a pharmaceutical composition for preventing and/or treating a cancer, in particular to a pharmaceutical composition containing a CDK4/6 inhibitor compound of Formula (I) or a pharmaceutically acceptable salt thereof in combination with a second therapeutic agent or a pharmaceutically acceptable salt thereof at a fixed ratio or a fixed dose, a use of the above pharmaceutical composition in preparation of a drug for preventing and/or treating a cancer, and a kit containing the pharmaceutical composition.

### CROSS-REFERENCE TO RELATED APPLICATION

The present application is a National Stage of International Patent Application No. PCT/CN2021/143483, filed on Dec. 31, 2021, and claims priority to and interest of patent application No. 202011632660.3, filed to the China National Intellectual Property Administration on Dec. 31, 2020, the disclosure of which is hereby incorporated by reference in its entirety.

### TECHNICAL FIELD

The present disclosure belongs to the technical field of medicines, and specifically relates to a pharmaceutical composition for preventing and/or treating a cancer, in particular to a pharmaceutical composition containing a compound of Formula (I) or a pharmaceutically acceptable salt thereof in combination with a second therapeutic agent or a pharmaceutically acceptable salt thereof at a fixed ratio or a fixed dose, and a use of the pharmaceutical composition for treating the cancer.

### BACKGROUND

A breast cancer is the most common malignant tumor among women, and there are about 2 million new breast cancer patients worldwide every year. The breast cancer is a type of tumor with high heterogeneity at the molecular level which has significant differences in tissue morphology, immune phenotype, biological behavior, and treatment response. Based on the expression of three receptors: the estrogen receptor (ER), the progesterone receptor (PR) and the human epidermal growth factor receptor-2 (Her2), the breast cancer is divided into three subtypes. The estrogen receptor positive (ER+) breast cancer is the most common molecular subtype of the breast cancer, and the incidence rate of population is about 65%-70%. In the breast cancer, it has the best prognosis, and there are more patients in the early stage. Compared with other molecular subtypes of the breast cancer, the ER+ breast cancer has much better prognosis, but it still has a 5-year recurrence and metastasis rate of about 15-30%. The human epidermal growth factor receptor-2 (HER2) molecule is an independent factor with poorer prognosis in the breast cancer, and about 20%-30% of Chinese breast cancer patients have HER2 gene amplification/over-expression. The HER2 over-expression is typically associated with invasive and metastatic forms of the breast cancer, and these invasive and metastatic forms of the breast cancer have a high recurrence rate and/or are associated with the poor prognosis of patients.

The general treatment goal of all ER/PR positive metastatic breast cancer patients is to prolong the survival period and improve the quality of life. This may be achieved by surgical intervention and drug therapy (if possible). Endocrine (anti-estrogen) drugs are usually initially used and maintained until the drug resistance appears. Their use avoids the toxicity based on chemotherapy regimens, but the toxicity and drug resistance of the drug itself still need to be well addressed.

ER drugs are mainly divided into a selective estrogen receptor modulator (SERM), a selective estrogen receptor degrader (SERD), an aromatase inhibitor etc. The SERD drugs on the market include fulvestrant (Astrazeneca), and the SERDs under research include AZD-9496 (Astrazeneca), RAD1901 (Eisai), and ZB-716 (Louisiana University).

Almost all the functional effects of oncogenes and tumor suppressor genes may eventually converge to the cell cycle, and regulating or blocking the cell cycle is one way to treat tumors. At present, many molecules related to the cell cycle regulation have already been discovered, in which Cyclin-Dependent-Kinases (CDKs) are core molecules of the cell cycle regulatory network. In CDK subtypes involved in the cell cycle, CDK4/6 plays an irreplaceable role. The specific activation of CDK4/6 is closely related to the proliferation of some tumors, and about 80% of human tumors exhibit abnormalities in the cyclin D-CDK4/6-INK4-Rb pathway. The CDK4/6 inhibitors currently on the market include PD0332991 (Palbociclib, Ibrance) developed by Pfizer, LY2835219 (Abemaciclib, Verzenio) of Lilly, and LEE011 (Ribociclib, Kisqali) of Novartis.

Like other kinase inhibitors used for treating cancers, the effective use of the CDK4/6 inhibitor is also limited by the drug resistance. The present disclosure provides a tumor treatment composition that may effectively solve the problem of the drug resistance and has the relatively low toxicity, and the drug resistance is mainly aimed at endocrine and CDK inhibition therapies.

### SUMMARY

In one aspect, the present disclosure provides a pharmaceutical composition for preventing and/or treating a cancer, and it contains a compound of Formula (I) below or a pharmaceutically acceptable salt thereof, and at least one second therapeutic agent or a pharmaceutically acceptable salt thereof.

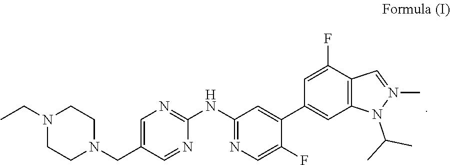

The second therapeutic agent is a selective estrogen receptor degrader, wherein the weight ratio of active ingredients of the compound of Formula (I) or the pharmaceutically acceptable salt thereof to that of the second therapeutic agent or the pharmaceutically acceptable salt thereof is 1:1-3000:1.

In some embodiments, the weight ratio of the active ingredients of the compound of Formula (I) or the pharmaceutically acceptable salt thereof to that of the second therapeutic agent or the pharmaceutically acceptable salt thereof is 1:1-2672:1.

In some embodiments, the weight ratio of the active ingredients of the compound of Formula (I) or the pharmaceutically acceptable salt thereof to that of the second therapeutic agent or the pharmaceutically acceptable salt thereof is 5.2:1-2672:1.

In some embodiments, the weight ratio of the active ingredients of the compound of Formula (I) or the pharmaceutically acceptable salt thereof to that of the second therapeutic agent or the pharmaceutically acceptable salt thereof is 1:1-800:1.

In some embodiments, the weight ratio of the active ingredients of the compound of Formula (I) or the pharmaceutically acceptable salt thereof to that of the second therapeutic agent or the pharmaceutically acceptable salt thereof is 1:1-500:1.

In some embodiments mentioned above, the second therapeutic agent is fulvestrant.

In some embodiments, the second therapeutic agent is fulvestrant, and the weight ratio of active ingredients of the compound of Formula (I) or the pharmaceutically acceptable salt thereof to that of the fulvestrant or a pharmaceutically acceptable salt thereof is 10:1-1200:1.

In some embodiments, the second therapeutic agent is fulvestrant, and the weight ratio of the active ingredients of the compound of Formula (I) or the pharmaceutically acceptable salt thereof to that of the fulvestrant or the pharmaceutically acceptable salt thereof is 10:1-800:1.

In some embodiments, the second therapeutic agent is fulvestrant, and the weight ratio of the active ingredients of the compound of Formula (I) or the pharmaceutically acceptable salt thereof to that of the fulvestrant or the pharmaceutically acceptable salt thereof is 10:1-600:1.

In some embodiments, the second therapeutic agent is fulvestrant, and the weight ratio of the active ingredients of the compound of Formula (I) or the pharmaceutically acceptable salt thereof to that of the fulvestrant or the pharmaceutically acceptable salt thereof is 10:1-400:1,

In some embodiments, the second therapeutic agent is fulvestrant, and the weight ratio of the active ingredients of the compound of Formula (I) or the pharmaceutically acceptable salt thereof to that of the fulvestrant or the pharmaceutically acceptable salt thereof is 10:1-200:1.

In some embodiments, the second therapeutic agent is fulvestrant, and the weight ratio of the active ingredients of the compound of Formula (I) or the pharmaceutically acceptable salt thereof to that of the fulvestrant or the pharmaceutically acceptable salt thereof is 20:1-150:1.

In some embodiments, the second therapeutic agent is fulvestrant, and the weight ratio of the active ingredients of the compound of Formula (I) or the pharmaceutically acceptable salt thereof to that of the fulvestrant or the pharmaceutically acceptable salt thereof is 30:1-150:1.

In some embodiments, the second therapeutic agent is fulvestrant, and the weight ratio of the active ingredients of the compound of Formula (I) or the pharmaceutically acceptable salt thereof to that of the fulvestrant or the pharmaceutically acceptable salt thereof is 40:1-150:1.

In some embodiments, the second therapeutic agent is fulvestrant, and the weight ratio of the active ingredients of the compound of Formula (I) or the pharmaceutically acceptable salt thereof to that of the fulvestrant or the pharmaceutically acceptable salt thereof is 20:1-120:1.

In some embodiments, the second therapeutic agent is fulvestrant, and the weight ratio of the active ingredients of the compound of Formula (I) or the pharmaceutically acceptable salt thereof to that of the fulvestrant or the pharmaceutically acceptable salt thereof is 30:1-120:1.

In some embodiments, the second therapeutic agent is fulvestrant, and the weight ratio of the active ingredients of the compound of Formula (I) or the pharmaceutically acceptable salt thereof to that of the fulvestrant or the pharmaceutically acceptable salt thereof is 40:1-120:1.

In some embodiments, the second therapeutic agent is fulvestrant, and the weight ratio of the active ingredients of the compound of Formula (I) or the pharmaceutically acceptable salt thereof to that of the fulvestrant or the pharmaceutically acceptable salt thereof is 20:1-100:1.

In some embodiments, the second therapeutic agent is fulvestrant, and the weight ratio of the active ingredients of the compound of Formula (I) or the pharmaceutically acceptable salt thereof to that of the fulvestrant or the pharmaceutically acceptable salt thereof is 30:1-100:1.

In some embodiments, the second therapeutic agent is fulvestrant, and the weight ratio of the active ingredients of the compound of Formula (I) or the pharmaceutically acceptable salt thereof to that of the fulvestrant or the pharmaceutically acceptable salt thereof is 40:1-100:1.

In some embodiments, the second therapeutic agent is fulvestrant, and the weight ratio of the active ingredients of the compound of Formula (I) or the pharmaceutically acceptable salt thereof to that of the fulvestrant or the pharmaceutically acceptable salt thereof is 1:1-150:1, preferably 10:1-20:1, 20:1-30:1, 30:1-40:1, 40:1-50:1, 50:1-60:1, 60:1-70:1, 70:1-80:1, 80:1-90:1, 90:1-100:1, 100:1-110:1, 110:1-120:1, 120:1-130:1, 130:1-140:1 or 140:1-150:1.

In some embodiments, the second therapeutic agent is fulvestrant, and the weight ratio of the active ingredients of the compound of Formula (I) or the pharmaceutically acceptable salt thereof to that of the fulvestrant or the pharmaceutically acceptable salt thereof is 107.52:1, 94.08:1, 80.64:1, 67.2:1, 62.72:1, 53.76:1, 47.04:1, 40.32:1, 33.6:1, 31.36:1, 26.88:1, 23.52:1, 20.16:1, 16.8:1, 15.68:1, 13.44:1 and 11.76:1.

In some embodiments, the second therapeutic agent is fulvestrant, and the weight ratio of the active ingredients of the compound of Formula (I) or the pharmaceutically acceptable salt thereof to that of the fulvestrant or the pharmaceutically acceptable salt thereof is 115.2:1, 100.8:1, 86.4:1, 72:1, 67.2:1, 57.6:1, 50.4:1, 43.2:1, 36:1, 33.6:1, 28.8:1, 25.2:1, 21.6:1, 18:1, 16.8:1, 14.4:1, and 12.6:1.

In some embodiments, the daily dose of the active ingredients of the compound of Formula (I) or the pharmaceutically acceptable salt thereof is 200-2000 mg.

In some embodiments, the daily dose of the active ingredients of he compound of Formula (I) or the pharmaceutically acceptable salt thereof is 200-1000 mg.

In some embodiments, the daily dose of the active ingredients of the compound of Formula (I) or the pharmaceutically acceptable salt thereof is 400-1000 mg.

In some embodiments, the daily dose of the active ingredients of the compound of Formula (I) or the pharmaceutically acceptable salt thereof is 960 mg, 840 mg, 720 mg, 600 mg, 560 mg, 480 mg, or 420 mg.

In some embodiments, the administration frequency of the compound of Formula (I) or the pharmaceutically acceptable salt thereof is once a day, twice a day, or three times a day.

In some embodiments, the administration frequency of the second therapeutic agent or the pharmaceutically acceptable salt thereof is once a month, once every 28 days, once every half month, or once every 14 days.

In some embodiments, the amount per dose of the active ingredients of the second therapeutic agent or the pharmaceutically acceptable salt thereof is 1-1000 mg, such as 10-1000 mg, such as 100-1000 mg, such as 250-1000 mg.

In some embodiments mentioned above, the second therapeutic agent is fulvestrant.

In some embodiments, the second therapeutic agent is fulvestrant, and the amount per dose of the active ingredients of the fulvestrant or the pharmaceutically acceptable salt thereof is 250 mg or 500 mg.

In some embodiments, the second therapeutic agent is fulvestrant, and the amount per dose of the active ingredients of the fulvestrant or the pharmaceutically acceptable salt thereof is 250 mg or 500 mg, and the administration frequency is once a month, once every 28 days, once a half month, or once every 14 days.

In some embodiments, the compound of Formula (I) is administered simultaneously or sequentially with the second therapeutic agent.

In some embodiments, the administration route of the compound of Formula (I) or the pharmaceutically acceptable salt thereof is oral administration.

In some embodiments, the administration mode of the second therapeutic agent or the pharmaceutically acceptable salt thereof is oral administration or injection administration.

In another aspect, the present disclosure further provides a pharmaceutical formulation containing the aforementioned pharmaceutical composition and one or more pharmaceutically acceptable excipients, and the pharmaceutical formulation may be any one pharmaceutically acceptable formulation. The pharmaceutically acceptable excipients are substances that are non-toxic, compatible with the active ingredients, and otherwise biologically suitable for living organisms. The selection of specific excipients may depend on the administration mode used to treat specific patients or the type and status of the disease.

In some embodiments, the above pharmaceutical formulation may be administered to a patient or a subject in need of such treatment by oral, parenteral, rectal or transpulmonary administration and the like. While used for the oral administration, the pharmaceutical composition may be made into an oral formulation, for example, a conventional oral solid formulation, such as tablets, capsules, pills, granules and the like; and it may also be made into an oral liquid formulation, such as an oral solution, an oral suspension, syrup and the like. While used for the parenteral administration, the above pharmaceutical formulations may also be made into an injection, including an injection solution, a sterile powder for injection, and a concentrated solution for injection. While used for the rectal administration, the pharmaceutical composition may be made into a suppository or the like. While used for the transpulmonary administration, the pharmaceutical composition may be made into an inhalation preparation, an aerosol, a dry powder inhalation or a spray or the like.

In another aspect, the present disclosure further relates to the use of the aforementioned pharmaceutical composition in preparation of a medicament for preventing and/or treating a cancer, and the cancer is selected from the group consisting of brain tumor, lung cancer, squamous cell carcinoma, bladder cancer, gastric cancer, ovarian cancer, peritoneal cancer, pancreatic cancer, breast cancer, head and neck cancer, cervical cancer, endometrial cancer, rectal cancer, liver cancer, kidney cancer, esophageal adenocarcinoma, esophageal squamous cell carcinoma, prostate cancer, female genital tract cancer, lymphoma, neurofibroma, thyroid cancer, bone cancer, skin cancer, brain cancer, colon cancer, testicular cancer, gastrointestinal stromal tumor, prostate tumor, mast cell tumor, multiple myeloma, melanoma, glioma or sarcoma, preferably the breast cancer.

In some embodiments, the cancer is HR⁺, HER2⁻ breast cancers.

In some embodiments, the cancer is postmenopausal HR⁺, HER2⁻ breast cancers.

In some embodiments, the cancer is locally advanced or metastatic breast cancers.

In some embodiments, the cancer is HR⁺, HER2⁻ advanced or metastatic breast cancers.

In some embodiments, the cancer is HR⁺, HER2⁻ advanced or metastatic breast cancers that progress after a single endocrine treatment.

In another aspect, the present disclosure further relates to the use of a combination of a therapeutic effective amount of the compound of Formula (I) or the pharmaceutically acceptable salt thereof and the fulvestrant or the pharmaceutically acceptable salt thereof in preparation of a medicament for preventing and/or treating a cancer, and the cancer is selected from a group consisting of brain tumor, lung cancer, squamous cell carcinoma, bladder cancer, gastric cancer, ovarian cancer, peritoneal cancer, pancreatic cancer, breast cancer, head and neck cancer, cervical cancer, endometrial cancer, rectal cancer, liver cancer, kidney cancer, esophageal adenocarcinoma, esophageal squamous cell carcinoma, prostate cancer, female genital tract cancer, lymphoma, neurofibroma, thyroid cancer, bone cancer, skin cancer, brain cancer, colon cancer, testicular cancer, gastrointestinal stromal tumor, prostate tumor, mast cell tumor, multiple myeloma, melanoma, glioma or sarcoma, preferably the breast cancer.

In some embodiments, the cancer is HR⁺, HER2⁻ breast cancers.

In some embodiments, the cancer is postmenopausal HR⁺, HER2⁻ breast cancers.
In some embodiments, the cancer is locally advanced or metastatic breast cancers.

In some embodiments, the cancer is HR⁺, HER2⁻ advanced or metastatic breast cancers.

In some embodiments, the cancer is HR⁺, HER2⁻ advanced or metastatic breast cancers that progress after a single endocrine treatment.

Further, the present disclosure further relates to the use of a pharmaceutical formulation containing the aforementioned pharmaceutical composition in preparation of a drug for preventing and/or treating a cancer.

In another aspect, the present disclosure further provides a kit, and it contains the aforementioned pharmaceutical composition and an instruction for explaining how the pharmaceutical composition or the compound of Formula (I) and the second therapeutic agent contained therein are administered.

In another aspect, the present disclosure further provides a method for treating a cancer, and the method includes administering an effective amount of the aforementioned pharmaceutical composition or a pharmaceutical formulation containing the pharmaceutical composition to a patient in need; and the cancer is as described above.

In some embodiments, the compound of Formula (I) or the pharmaceutically acceptable salt thereof is administered in combination with the second therapeutic agent or the pharmaceutically acceptable salt thereof according to a specific dosage regimen.

In some embodiments, the compound of Formula (I) or the pharmaceutically acceptable salt thereof is administered orally at a dose of 400-1000 mg per day.

In some embodiments, the compound of Formula (I) or the pharmaceutically acceptable salt thereof is administered orally at a dose of 960 mg, 840 mg, 720 mg, 600 mg, 560 mg, 480 mg, or 420 mg per day.

In some embodiments, the administration frequency of the compound of Formula (I) or the pharmaceutically acceptable salt thereof is once a day or twice a day.

In some embodiments, the second therapeutic agent is fulvestrant, and the fulvestrant or the pharmaceutically acceptable salt thereof is administered at a dose of 250 mg or 500 mg on the 1st, 15th, and 29th days, and thereafter once a month or once every 28 days.

In the present disclosure, unless otherwise stated, scientific and technical terms used herein have the meanings commonly understood by those skilled in the art. However, in order to understand the present disclosure better, definitions of some terms are provided below. While the definition and explanation of the terms provided by the present disclosure do not conform to the meanings commonly understood by those skilled in the art, the definition and explanation of the terms provided by the present disclosure shall prevail.

The HR positive (HR⁺) mentioned in the present disclosure means that the expression of estrogen receptor (ER) and/or progesterone receptor (PR) is positive, namely HR⁺ includes ER⁺, PR⁺, or ER⁺/PR⁺.

The HER2⁻ mentioned in the present disclosure means that the expression of human epidermal growth factor receptor 2 (HER2) is negative.

The “weight ratio of active ingredients” mentioned in the present disclosure may be the daily dose ratio of drugs, the weekly dose ratio, the monthly dose ratio or the dose ratio for each administration cycle. If the administration frequencies of two drugs are different, the same standard is selected for conversion, as to obtain the weight ratio of the active ingredients.

The “daily dose” mentioned in the present disclosure may be the actual daily dose or the daily dose obtained by the conversion. For example, if the administration frequency is 360 mg twice a day, the daily dose is 720 mg; and for example, if the administration frequency is 300 mg once every 15 days, the daily dose is 20 mg.

The “second therapeutic agent” mentioned in the present disclosure refers to an agent that has certain preventive and/or therapeutic effects on tumors, including but not limited to a mitotic inhibitor, an alkylating agent, a selective estrogen receptor modulator, a selective estrogen receptor degrader, a DNA intercalating agent, an anti-tumor antibiotic, a growth factor inhibitor, a vitamin A receptor-like modulator, a topoisomerase inhibitor, a hormone drug, an angiogenesis inhibitor etc. The “effective amount” refers to a drug dose that may prevent, alleviate, delay, suppress, or cure symptoms of a subject. The amount of the administration dose is related to the drug administration mode, pharmacokinetics of agents, severity of diseases, and individual physical signs (gender, weight, height, and age) of the subject etc.

The “pharmaceutical composition” in the present disclosure includes a composition formed by a same compound formulation prepared by the active ingredients such as the compound of Formula (I) and the second therapeutic agent, and also includes a composition formed by a single formulation prepared separately by the active ingredients such as the compound of Formula (I) and the second therapeutic agent.

The “simultaneous administration” of the compound of Formula (I) and the second therapeutic agent mentioned in the present disclosure includes that the compound of Formula (I) and the second therapeutic agent are made into the same compound formulation and administered, or the compound of Formula (I) and the second therapeutic agent are separately made into the formulations and administered simultaneously.

The “separate administration” of the compound of Formula (I) and the second therapeutic agent mentioned in the present disclosure means that the compound of Formula (I) and the second therapeutic agent are separately made into the formulations, and administered sequentially separately according to respective preparation administration modes.

The ‘pharmaceutically acceptable salt’ mentioned in the present disclosure refers to salts formed by acidic functional groups (such as —COOH, —OH, and —SO₃H) present in compounds and appropriate inorganic or organic cations (alkalis), including salts formed with alkali metals or alkaline earth metals, ammonium salts, and salts formed with nitrogen-containing organic alkalis; and salts formed by alkaline functional groups (such as —NH₂) present in the compounds and appropriate inorganic or organic anions (acids), including salts formed with inorganic acids or organic acids (such as a carboxylic acid).

Beneficial effects of the Present Disclosure
1. The pharmaceutical composition of the present disclosure has the excellent anti-tumor effects, especially the significant breast cancer therapeutic effect.
2. The pharmaceutical composition of the present disclosure may effectively solve the problems of drug resistance to the endocrine therapy and the long-term use of the CDK inhibitor.
3. The pharmaceutical composition of the present disclosure is low in toxicity or non-toxic, and relatively fewer in side effects.

### BRIEF DESCRIPTION OF THE DRAWINGS

FIG. 1 is a cell proliferation inhibition curve of a combined use of a compound of Formula (I) and fulvestrant at a fixed ratio, wherein the horizontal coordinate is a logarithm of a test concentration (c) of the compound of Formula (I), namely Ig c; and the vertical coordinate is a suppression rate (%)

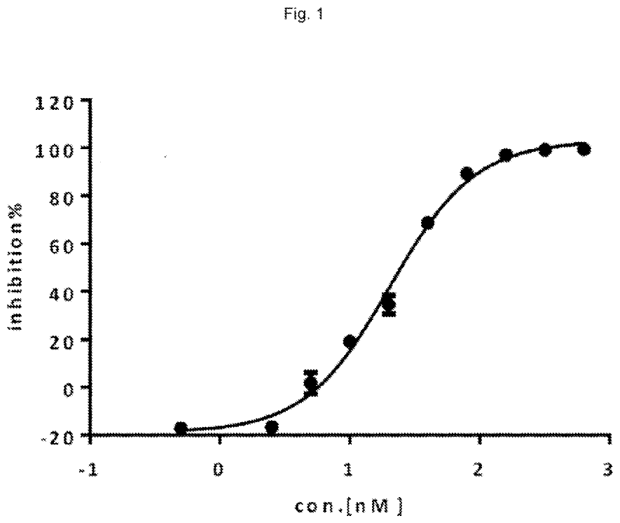

### DETAILED DESCRIPTION OF THE EMBODIMENTS

### Experimental Schemes

Exemplary experimental schemes for some compounds of the present disclosure are provided below, to show the beneficial activity and technical effects of the compounds of the present disclosure. However, it should be understood that the following experimental schemes are only examples of the content of the present disclosure, and are not limitations to the scope of the present disclosure.

### EXPERIMENTAL EXAMPLE 1

### In Vitro Cytological Inhibition Activity of Compound of Formula (I) of Present Disclosure and Fulvestrant Administered Separately or in Combination

Test Samples:

The compound of Formula (I) of the present disclosure: prepared according to a method in prior art.

Fulvestrant: purchased or prepared according to a method in prior art.

The abbreviations for the following experiments represent the meanings as follows:

| Symbol | Name | Symbol | Name |
|---|---|---|---|
| DMSO | Dimethyl sulfoxide | BrdU-Eu | Europium-labeled |
| | | | bromodeoxyuridine |
| FBS | Fetal bovine serum | Flu | Fluorescence value |
| CI | Combination index | MEM | Minimum essential medium |
| BrdU | Bromodeoxyuridine | MCF7 | Human breast cancer cell |

Experimental method: Cell proliferation detection using BrdU method (BrdU cell proliferation test kit, PerkinElmer Company)
1. Reagent and compound preparation
1.1. Preparation of 1-fold washing solution:

A washing solution with a mother liquor concentration of 25 times was diluted into 1-fold washing solution with ultrapure water.
1.2. Preparation of 1-fold detection antibody solution:

A BrdU-Eu detection antibody with a mother liquor concentration of 200 times was diluted into 1-fold detection antibody solution with a detection antibody diluent.
1.3. 100-fold BrdU labeling solution:

A BrdU stock solution with a mother liquor concentration of 1000 times was diluted into 100-fold BrdU labeling solution with a culture medium corresponding to cells.
1.4. Preparation of test compounds:

The mother liquor of the compound of Formula (I) was diluted to 0.256 mM with DMSO, and then diluted in a double continuous gradient for 9 concentrations from 0.256 mM; the mother liquor of the fulvestrant was diluted to 1.28 μM with DMSO, and then diluted in the double continuous gradient for 9 concentrations from 1.28 μM; and the mother liquor of the fulvestrant was diluted to 80 nM and 160 nM with DMSO.

Single compound: the gradient diluent of the compound of Formula (I) or the fulvestrant is respectively mixed with DMSO uniformly at 1:1.

Combination compound of a fixed ratio: the gradient diluents of the compound of Formula (I) and the fulvestrant are mixed uniformly at 1:1.

Combination compound of a fixed concentration: the compound of Formula (I) is respectively mixed with 80 nM or 160 nM of the fulvestrant uniformly at 1:1.
1.5. Preparation of culture medium:

MCF7 medium: MEM+10% FBS+1% bispecific antibody+10 μg/mL human recombinant insulin
2. Test steps
2.1. Cells were routinely cultured until the cell fusion degree was 80%-90%, and the cells were collected after trypsin digestion and centrifuged. Then resuspended with a corresponding culture medium, counted and adjustment-inoculated in a 96-well plate, the MCF7 cells were inoculated according to 8000 cells/well/100 μL, placed and cultured in a 37° C. cell incubator, and after the cells were adhered to a wall, it was replaced with a serum-free medium.
2.2. 99 μL of a growth medium containing 20% FBS was supplemented per well, and then 1 μL of a diluted 200× to-be-tested substance was added to the wells, the final concentration of the to-be-tested substance was shown in the table below, and it is cultured in the 37° C. incubator for 48 hours.

Solvent control: 0.5% DMSO; and blank control: only the culture medium was added, no cells.

| | To-be-tested | | | | | | | | | |
|---|---|---|---|---|---|---|---|---|---|---|
| | substance | Final concentration (nM) | | | | | | | | |
| Single | Compound of | 640 | 320 | 160 | 80 | 40 | 20 | 10 | 5 | 2.5 |
| | Formula (I) | | | | | | | | | |
| Single | Fulvestrant | 3.2 | 1.6 | 0.8 | 0.4 | 0.2 | 0.1 | 0.05 | 0.025 | 0.0125 |
| Combination | Compound of | 640 | 320 | 160 | 80 | 40 | 20 | 10 | 5 | 2.5 |
| Fixed ratio | Formula (I) | | | | | | | | | |
| | Fulvestrant | 3.2 | 1.6 | 0.8 | 0.4 | 0.2 | 0.1 | 0.05 | 0.025 | 0.0125 |
| Combination | Compound of | 640 | 320 | 160 | 80 | 40 | 20 | 10 | 5 | 2.5 |
| Fixed | Formula (I) | | | | | | | | | |
| concentration | Fulvestrant | 0.2 | 0.2 | 0.2 | 0.2 | 0.2 | 0.2 | 0.2 | 0.2 | 0.2 |
| | Compound of | 640 | 320 | 160 | 80 | 40 | 20 | 10 | 5 | 2.5 |
| | Formula (I) | | | | | | | | | |
| | Fulvestrant | 0.4 | 0.4 | 0.4 | 0.4 | 0.4 | 0.4 | 0.4 | 0.4 | 0.4 |

2.3. 2 μL of the 100-fold BrdU labeling solution was added per well, and incubated overnight in the cell incubator, then equilibrated at a room temperature for 0.5 hours, and the culture medium was sucked with a vacuum pump.
2.4. 100 μL of fixed solution was added per well, and incubated at the room temperature for 30 minutes, and the solution was discarded.
2.5. 100 μL of the 1-fold BrdU-Eu detection antibody solution was added per well, and incubated at the room temperature for 1 hour, the solution was discarded, and it was washed with the 1-fold washing solution at 250 μL/well for 5 times.
2.6. 200 μL of time-resolved immunofluorescence induction solution was added per well, and incubated at the room temperature for 15 minutes, and the fluorescence value (Flu value) was detected by a microplate reader.
3. Data processing
1) Cell inhibition rate (%)=1−(Flu_{test substance}−Flu_{blank control})/(Flu_{solvent control}−Flu_{blank control})×100%, Flu_{blank control}: a blank control value, and Flu_{solvent control}: a solvent control value.
2) Data was plotted with GraphPad Prism 6 software, to obtain a curve and an IC₅₀ value.
3) CompuSyn software was used to fit the combination index CI, and CI<1 indicates a synergistic effect between the two drugs.

Experimental Result:

TABLE 1
In vitro cytological activity of compound of Formula (I)
and fulvestrant administered separately or in combination

| | Cytological activity on |
|---|---|
| Test substance | MCF7 in vitro (IC₅₀, nM) |
| Compound of Formula (I) | 50.52 |
| Fulvestrant | 0.22 |
| Combination with fixed ratio | 20.38 |

TABLE 2
Combination of compound of Formula (I) and
fulvestrant in MCF7 in vitro cytology

| Concentration | | | Concentration | | |
|---|---|---|---|---|---|
| (nM) | Concentration | | (nM) | Concentration | |
| Compound of | (nM) | | Compound of | (nM) | |
| Formula (I) | Fulvestrant | CI | Formula (I) | Fulvestrant | CI |
| 640.0 | 0.2 | 0.0869 | 640.0 | 0.4 | 0.2170 |
| 320.0 | 0.2 | 0.1561 | 320.0 | 0.4 | 0.0890 |
| 160.0 | 0.2 | 0.3228 | 160.0 | 0.4 | 0.2139 |
| 80.0 | 0.2 | 0.5331 | 80.0 | 0.4 | 0.4323 |
| 40.0 | 0.2 | 0.6744 | 40.0 | 0.4 | 0.5930 |
| 20.0 | 0.2 | 0.5592 | 20.0 | 0.4 | 0.5695 |
| 10.0 | 0.2 | 0.6876 | 10.0 | 0.4 | 0.8332 |
| 5.0 | 0.2 | 0.7780 | 5.0 | 0.4 | 0.9107 |
| 2.5 | 0.2 | 0.5176 | 2.5 | 0.4 | 0.7492 |

Experimental Conclusion:

From the data in Tables 1 and 2, it may be seen that the compound of Formula (I) of the present disclosure has the high inhibitory activity on in vitro cells, and has the synergistic effect while used in combination with the fulvestrant.

### EXPERIMENTAL EXAMPLE 2

### In Vivo Pharmacodynamic Evaluation Test on Different Cancer Xenograft Models by Compound of Formula (I) of Present Disclosure and Fulvestrant Administered Separately or in Combination

Test Samples:

The compound of Formula (I) of the present disclosure: prepared according to a method in prior art.

Fulvestrant: purchased or prepared according to a method in prior art.

The abbreviations for the following experiments represent the meanings as follows:

| Symbol | Name | Symbol | Name |
|---|---|---|---|
| RTV | Relative tumor volume | TGI | Tumor growth inhibition rate |

Experimental Method:

1. Tumor Inoculating and Grouping

Estrogen was injected subcutaneously on one day before animal inoculation, twice a week, and 40 μg/20 μL for each time (estrogen specification: 4 mg/2 mL) until the experiment was ended. MCF7 cells resuspended by a serum-free medium were inoculated subcutaneously on the right rib of a Balb/c female nude mouse at 1.5×10⁷+matrigel/0.2 mL (cell: matrigel=1:1). While the tumor growed to a volume mean value of 163 mm³, it was grouped and administered. The group of the compound of Formula (I) was administered orally once a day at an dose of 30 mg/kg for 21 consecutive days; the group of the fulvestrant was administered subcutaneously once a week, 5 mg per animal, for 3 consecutive weeks; and while the compound of Formula (I) was used in combination with the fulvestrant, the same administration mode was used as while used alone.

2. Measurement and Experimental Indicator of Tumor

The tumor volume was measured twice a week with a vernier caliper, the long diameter and the short diameter of the tumor were measured, and its volume calculation formula is: volume=0.5×long diameter×short diameter². The relative tumor volume RTV and relative tumor volume increase ratio (T/C) were calculated according to measurement results. RTV=Vₜ/V₀, herein Vₜ is the mean tumor volume on the t-th day after being grouped and administered, and V₀ is the tumor volume mean on the day of being grouped and administered. T/C(%)=treatment group RTV/control group RTV×100%. Tumor growth inhibition rate TGI (%)=(1−T/C)×100%.

3. Statistical Analysis

SPSS statistical analysis software was used to perform inter-group statistical analysis on the tumor volume, and p<0.05 was considered to have a significant difference.

Experimental Result:

TABLE 3
Tumor growth inhibition results of compound of Formula (I) and fulvestrant
administered separately or in combination in MCF7 human xenograft model

| | Tumor volume before | Tumor volume at the end | | |
|---|---|---|---|---|
| Group | administration (mm³)^{a} | of administration (mm³)^{a} | TGI(%) | P^{b} |
| Solvent control group | 163 ± 17 | 468 ± 37 | − | − |
| Compound of Formula (I) | 162 ± 17 | 116 ± 7 | 76.5 | <0.001 |
| 30 mg/kg | | | | |
| Fulvestrant 5 mg/mouse | 163 ± 20 | 216 ± 44 | 55.9 | 0.001 |
| Compound of Formula (I) | 163 ± 20 | 85 ± 12 | 83.3 | <0.001 |
| 30 mg/kg + fulvestrant 5 | | | | |
| mg/mouse | | | | |

Note:
^{a}mean ± standard error; and
^{b}compared with the control group.

Experimental Conclusion:

From the data in Table 3, it may be seen that that the combination of the compound of Formula (I) and the fulvestrant has a significant inhibitory effect on the growth of MCF7 human breast cancer cells (TGI is 83.3%), and it is significantly better than that of the fulvestrant administered alone.

### EXPERIMENTAL EXAMPLE 3

### Clinical Trial of Drug Combination of Compound of Formula (I) of Present Disclosure and Fulvestrant in Treatment of Breast Cancer Patients

Test Samples:

The compound of Formula (I) of the present disclosure: prepared according to a method in prior art.

Fulvestrant: purchased or prepared according to a method in prior art.

Trial Inclusion Criteria:
1. Age: 18-70 years old (including 18 and 70 years old)
2. Patients with locally advanced or metastatic breast cancers who are hormone receptor positive (HR+) and HER2 negative (HER2−) confirmed by laboratory results, are not suitable for surgical resection or radiotherapy for the purpose of cure, and do not have clinical indications for chemotherapy, and who receive≤first line of endocrine therapy already and are allowed to receive 1 chemotherapy regimen.
3. The subjects must have at least one measurable lesion that meets the definition of RECISTv1.1.
4. The Eastern Cooperative Oncology Group (ECOG) score is 0-1.
5. All acute toxicity reactions from previous anti-cancer treatments or surgical procedures are remitted to a baseline or ≤level 1 (NCI-CTCAE v4.03 version, except for hair loss or other toxicity that researchers consider to be of no safety risk to the subjects).
6. At the time of enrollment, the organ functions of the subjects are good, and laboratory test data meets the following standards:
Blood routine: absolute neutrophil count≥2.0*10⁹/L (or greater than lower limit of normal value in a research center laboratory), platelet count≥100×10⁹/L, and hemoglobin≥100 g/L;
Liver function: serum total bilirubin≤1.5-fold upper limit of normal value (ULN), aspartate aminotransferase (AST) and alanine aminotransferase (ALT)≤1.5-fold ULN (if liver metastasis exists, AST and ALT≤3-fold ULN); and
Renal function: CrCl≥60 ml/min/1.73 m² (calculated according to a Cockcroft-Gault formula).
7. According to the judgment of the researchers, the expected lifespan of the subjects is ≥12 weeks.
8. Male or female subjects with fertility must agree to use effective contraceptive methods, such as a dual-barrier contraceptive method, a condom, an oral or an injectable contraceptive, and an intrauterine device, during the research period and within 3 months of the last research drug use.
9. Before the start of the research, the subjects must provide written informed consents.

Evaluation Criteria for Target Lesion

| | |
|---|---|
| Complete | All target lesions disappear, and the short diameter of all pathological lymph nodes |
| remission | (including a target nodule and a non-target nodule) must be reduced to <10 mm. |
| (CR) | |
| Partial | The sum of baseline limited diseases (LD) is used as a reference value, and the |
| remission (PR) | sum of LD of the target lesion is reduced by at least 30%. |
| Stable disease | The minimum sum of LD recorded since the start of treatment is used as a |
| (SD) | reference value, and the target lesion is not significantly reduced to conform PR or |
| | not significantly increased to conform PD. |
| Progressive | The minimum sum of LD recorded since the start of treatment is used as the |
| disease (PD) | reference value (including a case that the baseline value is the minimum value), |
| | the sum of LD of the target lesion is increased by at least 20%, and the absolute |
| | value is increased by at least 5 mm. (Note: the appearance of a new lesion may |
| | also be considered as progression) |

Administration Regimen:

The subjects were administered with the compound of Formula (I) and the fulvestrant.

Herein, the administration mode of the compound of Formula (I) was: 360 mg, administered orally twice a day. Every 28 days was a treatment cycle.

The administration mode of the fulvestrant was: 500 mg, injected intramuscularly on the first day of the first cycle (C1D1), the fifteenth day of the first cycle (C1D15), and the first day of each cycle (CnD1) subsequently. Every 28 days was a treatment cycle.

Test Results:

TABLE 4
Tumor inhibitory effect of drug combination of compound
of Formula (I) and fulvestrant on subjects

| | Tumor | | Tumor | | Compre- |
|---|---|---|---|---|---|
| | size before | | size after | Tumor | hensive |
| | combined | Treatment | combined | change | evaluation |
| Subject | therapy | cycle | therapy | ratio | result |
| 1 | Target lesion | 2 | Target lesion | +22% | PD |
| | 41 mm | | 50 mm | | |
| 2 | Target lesion | 3 | Target lesion | −50% | PR |
| | 20 mm | | 10 mm | | |
| 3 | Target lesion | 2 | Target lesion | 0 | SD |
| | 10 mm | | 10 mm | | |
| 4 | Target lesion | 2 | Target lesion | −34.8% | PR |
| | 23 mm | | 15 mm | | |
| 5 | Target lesion | 2 | Target lesion | −5.3% | SD |
| | 60.4 mm | | 57.2 mm | | |

Experimental Conclusion:

From the data in Table 4, it may be seen that after undergoing the first stage of 2-3 cycles of the combined therapy, the lesions of the subjects are significantly reduced already, and the lesions of some sensitive patients are even reduced by 50%. Two of the five subjects achieve PR, and other two achieve SD. The benefits from the combined therapy are significant.

### EXPERIMENTAL EXAMPLE 4

### Clinical Trial of Drug Combination of Compound of Formula (I) of Present Disclosure and Fulvestrant for Breast Cancer Patients with Disease Progression After Previous Endocrine Therapy

Test Samples:

The compound of Formula (I) of the present disclosure: prepared according to a method in prior art.

Fulvestrant: purchased or prepared according to a method in prior art.

Trial Inclusion Criteria:
1. Age: 18-70 years old (including 18 and 70 years old).
2. Patients who are HR+ and HER2− have the disease progression confirmed by laboratory results after receiving first line of the endocrine therapy in locally advanced, recurrent, or metastatic stages, and the patients are allowed to receive no more than 1 chemotherapy regimen.
3. The subjects must have at least one measurable lesion that meets the definition of RECISTv1.1.
4. The ECOG score is 0-1.
5. All acute toxicity reactions from previous anti-cancer treatment or surgical procedures are remitted to a baseline or ≤level 1 (NCE-CTCAE v4.03 version, except for hair loss or other toxicity that researchers consider to be of no safety risk to the subjects).
6. At the time of enrollment, the organ functions of the subjects are good, and laboratory test data meets the standards.
7. According to the judgment of the researchers, the expected lifespan of the subjects is ≥12 weeks.
8. Male or female subjects with fertility must agree to use effective contraceptive methods, such as a dual-barrier contraceptive method, a condom, an oral or an injectable contraceptive, and an intrauterine device, during the research period and within 3 months of the last research drug use.
9. Before the start of the research, the subjects must provide written informed consents.

Administration Regimen:

The subjects were administered with the compound of Formula (I) and the fulvestrant

Herein, the administration mode of the compound of Formula (I) was: 360 mg, administered orally twice a day. Every 28 days was a treatment cycle.

The administration mode of the fulvestrant was: 500 mg, injected intramuscularly on the first day of the first cycle (C1D1), the fifteenth day of the first cycle (C1D15), and the first day of each cycle (CnD1) subsequently. Every 28 days was a treatment cycle.

Test Results:

TABLE 5
Therapeutic response of compound of
Formula (I) combined with fulvestrant

| | Compound of Formula (I) combined |
|---|---|
| Item | with fulvestrant (N = 65) |
| CR | 0 |
| PR | 28(43.1) |
| SD | 32(49.2) |
| PD | 5(7.7) |
| DCR, n (%) | 60(92.3) |

Note:
DCR = disease control rate.

The compound of Formula (I) of the present disclosure combined with the fulvestrant has the excellent therapeutic effect on the breast cancer patients who have the disease progression after the previous endocrine therapy, DCR reaches 92.3%, and the benefits of the combined treatment are significant.

What is claimed is:

1. A pharmaceutical composition for preventing and/or treating a cancer, wherein the pharmaceutical composition comprises a therapeutic effective amount of a compound of Formula (I) or a pharmaceutically acceptable salt thereof, and at least one second therapeutic agent or a pharmaceutically acceptable salt thereof, 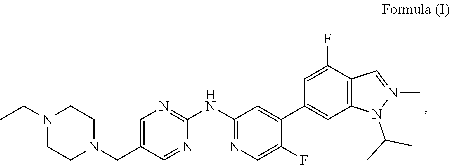
the second therapeutic agent is a selective estrogen receptor degrader, wherein a weight ratio of active ingredients of the compound of Formula (I) or the pharmaceutically acceptable salt thereof to that of the second therapeutic agent or the pharmaceutically acceptable salt thereof is 1:1-3000:1.

2. The pharmaceutical composition according to claim 1, wherein the weight ratio of the active ingredients of the compound of Formula (I) or the pharmaceutically acceptable salt thereof to that of the second therapeutic agent or the pharmaceutically acceptable salt thereof is 1:1-2672:1, preferably 1:1-800:1, and preferably 1:1-500:1.

3. The pharmaceutical composition according to claim 1, wherein the second therapeutic agent is fulvestrant, and the weight ratio of active ingredients of the compound of Formula (I) or the pharmaceutically acceptable salt thereof to that of the fulvestrant or a pharmaceutically acceptable salt thereof is 20:1-150:1, preferably 30:1-150:1, more preferably 40:1-150:1, and more preferably 40:1-50:1 and 80:1-90:1.

4. The pharmaceutical composition according to claim 1, wherein the second therapeutic agent is the fulvestrant, and the weight ratio of the active ingredients of the compound of Formula (I) or the pharmaceutically acceptable salt thereof to that of the fulvestrant or the pharmaceutically acceptable salt thereof is 107.52:1, 94.08:1, 80.64:1, 67.2:1, 62.72:1, 53.76:1, 47.04:1, 40.32:1, 33.6:1, 31.36:1, 26.88:1, 23.52:1, 21.6:1, 20.16:1, 18:1, 16.8:1, 15.68:1, 14.4:1, 13.44:1, 12.6:1, 11.76:1, 115.2:1, 100.8:1, 86.4:1, 72:1. 67.2:1, 57.6:1. 50.4:1, 43.2:1, 36:1, 33.6:1, 28.8:1 or 25.2:1.

5. The pharmaceutical composition according to claim 1, wherein a daily dose of the active ingredients of the compound of Formula (I) or the pharmaceutically acceptable salt thereof is 200-2000 mg, such as 200-1000 mg, such as 400-1000 mg; and wherein an amount per dose of the active ingredients of the second therapeutic agent or the pharmaceutically acceptable salt thereof is 1-1000 mg, such as 10-1000 mg, such as 100-1000 mg.

6. Use of the pharmaceutical composition according to claim 1 in preparation of a drug for preventing and/or treating a cancer, wherein the cancer is selected from a group consisting of brain tumor, lung cancer, squamous cell carcinoma, bladder cancer, gastric cancer, ovarian cancer, peritoneal cancer, pancreatic cancer, breast cancer, head and neck cancer, cervical cancer, endometrial cancer, rectal cancer, liver cancer, kidney cancer, esophageal adenocarcinoma, esophageal squamous cell carcinoma, prostate cancer, female genital tract cancer, lymphoma, neurofibroma, thyroid cancer, bone cancer, skin cancer, brain cancer, colon cancer, testicular cancer, gastrointestinal stromal tumor, prostate tumor, mast cell tumor, multiple myeloma, melanoma, glioma and sarcoma, preferably the breast cancer.

7. Use of a combination of a therapeutic effective amount of the compound of Formula (I) according to claim 1 or the pharmaceutically acceptable salt thereof and the fulvestrant or the pharmaceutically acceptable salt thereof in preparation of a drug for preventing and/or treating a cancer, wherein the cancer is selected from a group consisting of brain tumor, lung cancer, squamous cell carcinoma, bladder cancer, gastric cancer, ovarian cancer, peritoneal cancer, pancreatic cancer, breast cancer, head and neck cancer, cervical cancer, endometrial cancer, rectal cancer, liver cancer, kidney cancer, esophageal adenocarcinoma, esophageal squamous cell carcinoma, prostate cancer, female genital tract cancer, lymphoma, neurofibroma, thyroid cancer, bone cancer, skin cancer, brain cancer, colon cancer, testicular cancer, gastrointestinal stromal tumor, prostate tumor, mast cell tumor, multiple myeloma, melanoma, glioma and sarcoma, preferably the breast cancer.

8. The use according to claim 6, wherein the cancer is selected from HR⁺, HER2⁻ breast cancers.

9. The use according to claim 6, wherein the cancer is selected from locally advanced or metastatic breast cancers.

10. A kit, wherein the kit comprises the pharmaceutical composition according to claim 1 and an instruction manual for explaining how the pharmaceutical composition or the compound of Formula (I) contained therein and the second therapeutic agent are administered.

11. The use according to claim 7, wherein the cancer is selected from HR⁺, HER2⁻ breast cancers.

12. The use according to claim 7, wherein the cancer is selected from locally advanced or metastatic breast cancers.

13. A pharmaceutical formulation comprising the pharmaceutical composition according to claim 1 and one or more pharmaceutically acceptable excipients.

14. The pharmaceutical formulation according to claim 13, wherein the pharmaceutical formulation is administered by oral, parenteral, rectal or transpulmonary administration.

15. The pharmaceutical formulation according to claim 13, wherein the pharmaceutical formulation is an oral formulation, an injection, a suppository, an inhalation preparation, an aerosol, a dry powder inhalation or a spray.

16. A method for treating a cancer, comprising:
administering an effective amount of the pharmaceutical composition according to claim 1 or a pharmaceutical formulation containing the pharmaceutical composition according to claim 1 to a patient in need, and the cancer is selected from a group consisting of brain tumor, lung cancer, squamous cell carcinoma, bladder cancer, gastric cancer, ovarian cancer, peritoneal cancer, pancreatic cancer, breast cancer, head and neck cancer, cervical cancer, endometrial cancer, rectal cancer, liver cancer, kidney cancer, esophageal adenocarcinoma, esophageal squamous cell carcinoma, prostate cancer, female genital tract cancer, lymphoma, neurofibroma, thyroid cancer, bone cancer, skin cancer, brain cancer, colon cancer, testicular cancer, gastrointestinal stromal tumor, prostate tumor, mast cell tumor, multiple myeloma, melanoma, glioma and sarcoma, preferably the breast cancer.

17. The method according to claim 16, wherein the compound of Formula (I) or the pharmaceutically acceptable salt thereof is administered orally at a dose of 400-1000 mg per day.

18. The method according to claim 16, wherein the compound of Formula (I) or the pharmaceutically acceptable salt thereof is administered orally at a dose of 960 mg, 840 mg, 720 mg, 600 mg, 560 mg, 480 mg or 420 mg per day.

19. The method according to claim 16, wherein the administration frequency of the compound of Formula (I) or the pharmaceutically acceptable salt thereof is once a day or twice a day.

20. The method according to claim 16, wherein the second therapeutic agent is fulvestrant, and the fulvestrant or the pharmaceutically acceptable salt thereof is administered at a dose of 250 mg or 500 mg on the 1st, 15th, and 29th days, and thereafter once a month or once every 28 days.
